(19) United States
(12) Patent Application Publication
(10) Pub. No.: US 2008/0181872 A1
(43) Pub. Date: Jul. 31, 2008

(54) AAV VECTORS ENCODING SUPEROXIDE DISMUTASE

(76) Inventor: Mohammad Doroudchi, San Ramon, CA (US)

Correspondence Address: GENZYME CORPORATION;LEGAL DEPARTMENT, 15 PLEASANT ST CONNECTOR, FRAMINGHAM, MA, 01701-9322, US

(21) Appl. No.: 11/970,138

(22) Filed: Jan. 7, 2008

Related U.S. Application Data
(63) Continuation of application No. PCT/US2006/025980, filed on Jun. 30, 2006.
(60) Provisional application No. 60/697,450, filed on Jul. 7, 2005.

(51) Int. Cl. A61K 48/00; C12N 15/00; C12Q 1/70; A61P 25/00; A61P 19/00; A61P 3/00; A61P 27/02

(52) U.S. Cl. 424/93.2; 435/320.1; 435/5

(57) ABSTRACT

The invention relates to adeno-associated virus (AAV) vectors encoding superoxide dismutase (SOD), where the AAV vector encoding SOD (AAV-SOD) may be used to deliver the SOD gene to target cells. The target cells may be within a subject having a disease or condition for which delivery of SOD to the target cells provides a therapeutic benefit and/or a therapeutic effect on the subject. In another aspect, the invention relates to a model system for screening compounds for efficacy in treatment of amyotrophic lateral sclerosis (ALS). The model system may comprise a plurality of cells transduced with an AAV vector encoding an SOD gene; the transduced cells may exhibit a phenotypic change associated with ALS. The model system of the invention may be used to screen compounds for efficacy in treatment of ALS using

[symbol]

This application claims priority under 35 U.S.C. § 119(e) to U.S. Provisional Application No. 60/697,450, filed Jul. 7, 2005, the contents of which are herein incorporated by reference.

### TECHNICAL FIELD

The present invention relates to in vitro models for the screening of compounds for efficacy in treatment of amyotrophic lateral sclerosis (ALS). The present invention also relates to gene therapy vectors and methods.

### BACKGROUND

Neurodegenerative diseases present major public health issues. For example, amyotrophic lateral sclerosis (ALS) is a relentlessly progressive lethal disease that involves selective annihilation of motor neurons. Further information relating to ALS can be found in the Online Mendelian Inheritance in Man (OMIM) entry #105400, and in Rowland and Shneider (2001) Amyotrophic lateral sclerosis, New Eng. J. Med. 344: 1688-1700, the disclosures of which are hereby incorporated by reference in their entireties.

Mutations in genes encoding superoxide dismutase (SOD) have been associated with ALS. Three variants of SOD are known to be present in mammals. The cytoplasmic SOD is a copper zinc enzyme (Cu/Zn SOD) encoded by SOD1 (Weisiger and Fridovich (1973) J. Biol. Chem. 248, 4793-4796). As discussed infra, genetic defects in SOD1 have been associated with familial amyotrophic lateral sclerosis (fALS). Mitochondrial SOD (MnSOD) is associated with manganese and is encoded by SOD2. Li et al. ((1995) Nature Genetics 11, 376-381) describes a mutant mouse in which the gene-encoding SOD2 has been inactivated. A third mammalian SOD, encoded by SOD3 (Carlsson et al. (1995) Proc. Natl. Acad. Sci. USA 92, 6264-6268), also containing copper and zinc, is located largely extracellularly. Inactivation of this gene results in no overt phenotype.

Approximately 20% of fALS is linked to mutations in the SOD1 gene (Julien, J. P., Cell (2001) 104:581-591). Transgenic mice overexpressing the mutant SOD1 gene in which glycine 93 has been mutated to alanine (G93A) develop a dominantly inherited adult-onset paralytic disorder that has many of the clinical and pathological features of fALS (Gurney et al., Science (1994) 264:1772-1775). However, to date, the molecular mechanisms leading to motor neuron degeneration in ALS and most motor neuron diseases remain poorly understood, and there is currently no therapy available to prevent or cure ALS.

Methods of screening compounds effective in treating ALS are inefficient and labor intensive, hampering drug discovery. Although the ALS transgenic mice discussed above represent a useful in vivo method for assessing the efficacy of candidate compounds, experiments with mice are relatively expensive, time consuming and not well suited to high throughput screening. Other experiments rely on study of gene expression in post-mortem human samples, which are not abundant, and are not subject to controlled experimental manipulation.

An efficient in vitro model for ALS would facilitate rapid screening of potential therapeutic compounds. Compounds demonstrating beneficial effects in vitro could then be validated further with additional testing, for example using the transgenic mice discussed above. Existing in vitro methods, however, are unsuited to high throughput screening. For example, in one method, individual cells in primary culture are microinjected with a plasmid encoding a mutant SOD1 gene (e.g. G93A) to mimic the effects of over-expression of the same mutant gene in ALS. Such cells can then be treated with a compound of interest to determine whether the compound is able to reverse the phenotypic effect(s) associated with the over-expression of SOD1, such as formation of aggregates or inclusions. Microinjection, however, is labor intensive and can be performed on only a limited number of cells, making it difficult to obtain statistically robust results.

The need exists for a model system for screening compounds for efficacy in reversing or ameliorating the effects of ALS, which model should be amenable to high throughput screening and allow the evaluation of a statistically significant number of cells to determine the efficacy of compounds of interest.

Wild type SOD (not the mutant) may be useful as a therapeutic agent. A number of disorders are the result of oxidative stress, i.e. the presence of harmful reactive oxygen species (ROS) in cells, such as superoxide. See, e.g., Cash et al. (2004) Med. CheO. Rev. 1: 19-23. Superoxide dismutase catalyzes the conversion of superoxide to hydrogen peroxide and molecular oxygen. The hydrogen peroxide produced by SOD is subsequently converted to molecular oxygen and water by catalase, completing the conversion of superoxide to less reactive, and thus less damaging, forms of oxygen.

Antioxidants, such as vitamin A, vitamin C, glutathione, vitamin E, carotenes, lipoic acid, and coenzyme Q₁₀, can be administered to reduce the production and accumulation of such species, but such agents may not accumulate to effective levels within cells when administered systemically. As an alternative, sustained delivery of the enzyme SOD to such cells might help decrease the harmful effects of superoxide buildup.

The need exists for vectors and methods of therapy that are able to deliver an SOD gene to cells in a subject that can benefit from SOD activity. Such subjects include those suffering from disorders causing excess production or accumulation of superoxide, those exposed to environmental conditions causing excess superoxide production or accumulation, and even those subject to the cumulative oxidative damage associated with normal aging.

### SUMMARY OF THE INVENTION

In one aspect, the invention relates to adeno-associated virus (AAV) vectors encoding superoxide dismutase (SOD). In one embodiment the SOD is SOD1. In another embodiment the SOD1 gene contains a mutation associated with ALS, such as Gly93Ala.

In one embodiment the AAV vector encoding SOD (AAV-SOD) is used to deliver the SOD gene to target cells. In one embodiment the target cells are within a subject having a disease or condition for which delivery of SOD to the target cells provides a therapeutic benefit. In some embodiments, delivery of SOD results in a therapeutic effect on the subject. In other embodiments the disease or condition is selected from the group consisting of Parkinson's disease, Huntington's disease, degenerative eye diseases (e.g. macular degeneration, retinitis pigmentosa), Alzheimer's disease, rheumatoid arthritis, Crohn's disease, Peyronie's disease, ulcerative colitis, cerebral ischemia (stroke), myocardial infarct (heart attack), brain and/or spinal cord trauma, reperfusion damage, ALS, Down syndrome, cataracts, schizophrenia, epilepsy, human leukemia and other cancers, and diabetes.

In another aspect the invention relates to a model system for screening compounds for efficacy in treatment of amyotrophic lateral sclerosis (ALS) comprising a plurality of cells transduced with an AAV vector encoding a SOD1 gene containing a mutation associated with ALS, such as Gly93Ala. In various embodiments the AAV vector of the invention is derived from AAV-2, AAV-5 or AAV-6.

In one embodiment, the plurality of cells transduced with the AAV vector comprises at least 80% of the cells in the population in which they are found, for example a primary culture of cells from rodent spinal cord.

In some embodiments the transduced cells exhibit a phenotypic change associated with ALS. In other embodiments, one or more screened compounds reduce or ameliorate this phenotypic change.

In yet another aspect, the invention relates to methods of screening compounds for efficacy in treatment of ALS using a model system of the invention.

### BRIEF DESCRIPTION OF THE DRAWINGS

FIG. 1 is a schematic diagram of an rAAV vector for delivery of hSOD1-Gly93Ala, referred to as pVm-G93ASOD. Expression of hSOD1-Gly93Ala is driven by the chicken beta-actin promoter. The expression cassette is located between two AAV-2 ITR sequences. SOD1-Gly93Ala is also referred to herein as “mutant” SOD.

FIG. 2 is a schematic diagram of an rAAV vector for delivery of wild-type human SOD1, referred to as pVm-WTSOD. Expression of wtSOD1 is driven by the chicken beta-actin promoter. The expression cassette is located between two AAV-2 ITR sequences.

### DETAILED DESCRIPTION

The practice of the present invention will employ, unless otherwise indicated, conventional methods of virology, microbiology, cell and molecular biology and recombinant DNA techniques within the skill of the art. Such techniques are explained fully in the literature. See, e.g., Sambrook et al., Molecular Cloning: A Laboratory Manual (Current Edition); DNA Cloning. A Practical Approach, Vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijssen, ed.); Fundamental Virology, 2nd Edition, vol. I & II (BN. Fields and D. M. Knipe, eds.); Freshney, Culture of Animal Cells, A Manual of Basic Technique (Wiley-Liss, Third Edition); and Ausubel et al. (1991) Current Protocols in Molecular Biology (Wiley Interscience, NY).

All publications, patents, patent applications and database entries (including OMIM entries) cited herein are hereby incorporated by reference in their entireties.

The present invention relates to AAV vectors encoding SOD that can be used to create an in vitro model system for ALS, or as therapeutic agents.

SOD1 Mutations Associated with ALS

In the aspect of the invention relating to an ALS model system, the SOD gene encoded by the AAV vector comprise a mutation associated with ALS. “A mutation associated with ALS,” as used herein, refers to a mutation in an SOD gene that occurs with greater frequency in subjects having ALS than in subjects that do not have ALS. The amino acid sequence of wtSOD1 is presented at Table 1. Known mutations in SOD1 include Ala4Ser, Ala4Thr, Ala4Val (A4V; 147450.0012), Cys6Gly, Cys6Phe, Val7Glu, Leu8Val, Leu8Gln, Gly10Val, Gly12Arg, Val14Met, Val14Gly, Gly16Ala, Gly16Ser, Asn19Ser, Phe20Cys, Glu21Gly, Glu21Lys, Gln22Leu, Gly37Arg (G37R; 147450.0001), Leu38Arg, Leu38Val (L38V; 147450.0002), Gly41Asp (G41D; 147450.0004), Gly41Ser, His 43Arg, Phe45Cys, His46Arg (H46R; 147450.0013), Val47Phe, His48Arg, His48Gln, Glu49Lys, Thr54Arg, Cys57Arg, SerS9Ile, Asn65Ser, Leu67Arg, Gly72Cys, Gly72Ser, Asp76Tyr, Asp76Val, His80Ala, His80Arg, Leu84Val, Gly85Arg, Asn86Asp, Asn86Ser, Val87Met, Val87Ala, Ala89Thr, Ala89Val, Asp90Ala, Asp90Val, Gly93Ala (G93A; 147450.0008), Gly93Arg, Gly93Asp, Gly93Cys (G93C, 147450.0007), Gly93Ser, Gly93Val, Ala95Thr, Asp96Asn, Val97Met, Glu100Gly, Glu100Lys, Asp101Asn, Asp101Gly, Asp101His, Ile104Phe, Leu106Val, Ile113Thr (1113T; 147450.0011), Leu126Ter, Ser34Asn, Leu144Ser, and Ala145Thr. Numbers following mutations, when present, represent OMIM reference numbers for those mutations. SOD1 mutations are also disclosed at Rosen et al. (1993). Mutations in Cu/Zn superoxide dismutase gene are associated with familial amyotrophic lateral sclerosis, Nature 362: 59-62; Cudkowic et al. (1997) Epidemiology of mutations in superoxide dismutase in amyotrophic lateral sclerosis, Ann. Neurol. 41: 210-221; Belleroche et al. (1995). Familial amyotrophic lateral sclerosis/motor neuron disease (FALS): a review of current developments, J. Med. Genet. 32: 841-847. Although a number of mutations in SOD1 are disclosed herein, not all of these mutants will be useful in creating disease models for ALS.

TABLE 1
Amino Acid Sequence of wtSOD1

(SEQ ID NO. 1)

| | |
|---|---|
| 1 | ATKAVCVLKG DGPVQGIINF EQKESNGPVK VWGSIKGLTE |
| | GLHGFHVHEF |
| 51 | GDNTAGCTSA GPHFNPLSRK HGGPKDEERH VGDLGNVTAD |
| | KDGVADVSIE |
| 101 | DSVISLSGDH CIIGRTLVVH EKADDLGKGG NEESTKTGNA |
| | GSRLACGVIG |
| 151 | IAQ |

Adeno-Associated Virus Vectors

Adeno-associated virus (AAV) has been used with success to deliver genes for gene therapy and clinical trials in humans have demonstrated great promise (see, e.g., Kay et al., Nat. Genet. (2000) 24:257-261). As the only viral vector system based on a nonpathogenic and replication-defective virus, recombinant AAV virions have been successfully used to establish efficient and sustained gene transfer of both proliferating and terminally differentiated cells in a variety of tissues without detectable immune responses or toxicity (Bueler, H., Biol. Chem. (1999) 380:613-622).

The AAV genome is a linear, single-stranded DNA molecule containing about 4681 nucleotides. The AAV genome generally comprises an internal nonrepeating genome flanked on each end by inverted terminal repeats (ITRs). The ITRs are approximately 145 base pairs (bp) in length. The ITRs have multiple functions, including as origins of DNA replication, and as packaging signals for the viral genome. The internal nonrepeated portion of the genome includes two large open reading frames, known as the AAV replication (rep) and capsid (cap) genes. The rep and cap genes code for viral proteins that allow the virus to replicate and package into a virion. In particular, a family of at least four viral proteins are expressed from the AAV rep region, Rep 78, Rep 68, Rep 52, and Rep 40, named according to their apparent molecular weight. The AAV cap region encodes at least three proteins, VP1, VP2, and VP3.

AAV has been engineered to deliver genes of interest by deleting the internal nonrepeating portion of the AAV genome (i.e., the rep and cap genes) and inserting a heterologous gene between the ITRs. The heterologous gene is typically functionally linked to a heterologous promoter (constitutive, cell-specific, or inducible) capable of driving gene expression in the patient's target cells under appropriate conditions. Termination signals, such as polyadenylation sites, can also be included.

AAV is a helper-dependent virus; that is, it requires coinfection with a helper virus (e.g., adenovirus, herpesvirus or vaccinia), in order to form AAV virions in the wild. In the absence of coinfection with a helper virus, AAV establishes a latent state in which the viral genome inserts into a host cell chromosome, but infectious virions are not produced. Subsequent infection by a helper virus “rescues” the integrated genorne, allowing it to replicate and package its genome into an infectious AAV virion. While AAV can infect cells from different species, the helper virus must be of the same species as the host cell. Thus, for example, human AAV will replicate in canine cells coinfected with a canine adenovirus.

In a preferred embodiment of the present invention, a triple transfection method (described in detail in U.S. Pat. No. 6,001,650, incorporated by reference herein in its entirety) is used to produce rAAV virions because this method does not require the use of an infectious helper virus, enabling rAAV virions to be produced without any detectable helper virus present. This is accomplished by use of three vectors for rAAV virion production: an AAV helper function vector, an accessory function vector, and a rAAV expression vector. One of skill in the art will appreciate that the nucleic acid sequences encoded by these vectors can be provided on two or more vectors in various combinations.

The AAV helper function vector encodes the AAV helper function sequences (i.e., rep and cap), which function in trans for productive AAV replication and encapsidation. Preferably, the AAV helper function vector supports efficient AAV vector production without generating any detectable AAV virions containing functional rep and cap genes. An example of such a vector, pHLP19 is described in U.S. Pat. No. 6,001,650, incorporated herein by reference in its entirety. The rep and cap genes of the AAV helper function vector can be derived from any of the known AAV serotypes, as explained above. For example, the AAV helper function vector may have a rep gene derived from AAV-2 and a cap gene derived from AAV-6. One of skill in the art will recognize that other rep and cap gene combinations are possible, the defining feature being the ability to support rAAV virion production.

The accessory function vector encodes nucleotide sequences for non-AAV derived viral and/or cellular functions upon which AAV is dependent for replication, referred to herein as accessory functions. The accessory functions include those functions required for AAV replication, including, without limitation, those moieties involved in activation of AAV gene transcription, stage-specific AAV mRNA splicing, AAV DNA replication, synthesis of cap expression products, and AAV capsid assembly. Viral-based accessory functions can be derived from any of the well-known helper viruses such as adenovirus, herpesvirus (other than herpes simplex virus type-1), and vaccinia virus. In a preferred embodiment, the accessory function plasmid pLadeno5 is used. Details regarding pLadeno5 are described in U.S. Pat. No. 6,004,797, incorporated herein by reference in its entirety. This plasmid provides a complete set of adenovirus accessory functions for AAV vector production, but lacks the components necessary to form replication-competent adenovirus.

Recombinant AAV Expression Vectors

Recombinant AAV (rAAV) expression vectors are constructed using known techniques to provide operatively linked components including control elements (including a transcriptional initiation region), the SOD-encoding polynucleotide of interest and a transcriptional termination region. The resulting construct contains the operatively linked components bounded (5′ and 3′) with functional AAV ITR sequences.

In embodiments directed to a model system for the study of ALS, the control elements are selected to be functional in a mammalian neuronal cell. In embodiments directed to AAV-SOD vectors for therapeutic uses, the control elements are selected to be functional in the target cell or tissue of interest. Although tissue-specific and regulatable control elements are desirable in some embodiments of the present invention, other embodiments involve use of constitutive promoters.

The nucleotide sequences of AAV ITR regions are known. See, e.g., Kotin, R. M. (1994) Human Gene Therapy 5:793-801; Berns, K. I. “Parvoviridae and their Replication” in Fundamental Virology, 2nd Edition, (B. N. Fields and D. M. Knipe, eds.) for the AAV-2 sequence. AAV ITRs used in the vectors of the invention need not have a wild-type nucleotide sequence, and may be altered, e.g., by the insertion, deletion or substitution of nucleotides. Additionally, AAV ITRs may be derived from any of several AAV serotypes, including without limitation, AAV-1, AAV-2, AAV-3, AAV-4, AAV-5, AAV-6, AAV-7 and AAV-8, etc. AAV ITRs may also be derived, for example, from AAV variants isolated from murine, caprine or bovine sources. Furthermore, 5′ and 3′ ITRs that flank a selected nucleotide sequence in an AAV expression vector need not necessarily be identical or derived from the same AAV serotype or isolate, so long as they function as intended, i.e., to allow for excision and rescue of the sequence of interest from a host cell genome or vector, and to allow integration of the DNA molecule into the recipient cell genome when AAV Rep gene products are present in the cell.

Different AAV serotypes can be used to specifically target different cell types. For example, in various embodiments, the vectors of the present invention are derived from AAV-2, AAV-5, and AAV-6. For example, in primary rat motor neural cultures, AAV-6-derived vectors direct SOD expression primarily in neurons; AAV-2-derived vectors direct SOD expression in both neurons and glia; and AAV-5-derived vectors direct SOD expression primarily in glia. hSOD1-Gly93Ala causes morphological changes in motor neurons that are associated with pathology when delivered using an AAV-2-derived vector but does not cause pathology in motor neurons when delivered using an AAV-5-derived vector.

Therapeutic Uses of AAV-SOD

In various embodiments of the invention relating to therapeutic uses of rAAV-SOD, rAAV vectors are used to deliver the SOD1, SOD2 or SOD3 genes, or any combination thereof. In a particular embodiment, SOD1 is delivered.

The vectors described herein may be used to treat or prevent diseases or conditions associated with undesirable levels of ROS and free radicals, or oxidative stress generally. ROS are also generated as harmful side effects of some therapeutic drugs. See, e.g., Chan et al. (1996) Adv. Neurol 71: 271-279; DiGuiseppi and Fridovich (1984) Crit. Rev. Toxicol. 12: 315-342. Conditions that may be treated using the AAV-SOD vectors of the present invention include Parkinson's disease, Huntington's disease, degenerative eye diseases (e.g. macular degeneration, retinitis pigmentosa), Alzheimer's disease, rheumatoid arthritis, Crohn's disease, Peyronie's disease, ulcerative colitis, cerebral ischemia (stroke), myocardial infarct (heart attack), brain and/or spinal cord trauma, reperfusion damage, ALS, Down syndrome, cataracts, schizophrenia, epilepsy, human leukemia and other cancers, and diabetes.

Even subjects suffering from nothing more than the normal oxidative damage associated with aging may benefit from the vectors and methods of the present invention. A progressive rise of oxidative stress due to the formation of ROS and free radicals occurs during aging (see, e.g., Mecocci, P. et al. (2000) Free Radio. Biol. Med., 28: 1243-1248), as evidenced by an increase in the formation of lipid peroxidates in rat tissues (Erdincler, D. S., et al. (1997) Clin. Chim. Acta, 265: 77-84) and blood cells in elderly human patients (Congi, F., et al. (1995) Presse. Med., 24: 1115-1118). A recent review (Niki, E., Intern. Med. (2000) 39: 324-326) reported that increased tissue damage by ROS and free radicals could be attributed to the decreased levels of the antioxidative enzymes SOD and CAT that occur during aging. For example, transgenic animals generated by inserting extra SOD genes into the genome of mice were found to have decreased levels of ROS and free radical damage. Such animals also had an extended life span. More recent evidence indicated that administration of a small manganese porphyrin compound that mimics SOD activity led to a 44% extension of life span of the nematode worm Caenorhabditis elegans (S. Melow, et al. (2000) Science, 289: 1567-1569). Accordingly, the vectors and methods of the present invention may prevent and/or counteract the increased tissue damage and decreased life expectancy caused by the elevated levels of ROS and free radicals that accompany the aging process.

As used herein, “treatment” of ALS includes reversal of pre-existing damage, prevention of further damage, and slowing the progression of damage, each of which is a therapeutically desirable outcome. A therapeutic rAAV-SOD vector, or a compound discovered using an in vitro ALS model system of the present invention, need not reverse pre-existing damage to be considered therapeutically effective. Any treatment that at least slows the progression of ALS in a subject can be considered therapeutically effective.

In Vitro Model Systems of ALS to Screen for Therapeutic Compounds

As described in greater detail in the Examples, the AAV-SOD vectors of the present invention can be used to create an in vitro model system for ALS, which model system can be used to screen compounds for efficacy in the treatment or prevention of ALS. In one embodiment, a known mutant SOD1 associated with ALS (SOD1-Gly93Ala) is cloned into an AAV vector and a recombinant virion (rAAV-SOD1-Gly93Ala) is produced. rAAV-SOD1-Gly93Ala is then used to transduce a primary culture from a representative tissue, such as rat spinal cord or rat brain. The culture is then probed 3-5 days post-transfection to confirm that some of the neurons within the culture exhibit morphological changes associated with ALS, such as the formation of aggregates of SOD or vacuolization. Such cells are referred to herein as “ALS-like cells.” Such aggregates may be visualized by immunocytochemistry. In preferred embodiments, the percentage of ALS-like cells in the culture is high, such as 20, 30, 40, 50, 60, 70, 80, 90, or 95% or higher. The higher the percentage of ALS-like cells in a culture the greater the number of compounds that can be screened, or the greater the number of replicates for each individual compound, using the cells of the culture.

Screening is performed by exposing ALS-like cells to one or more compounds of interest and subsequently determining whether the phenotype of the ALS-like cells is altered in a way reflecting amelioration of ALS characteristics, such as a decrease in SOD aggregates. In some embodiments, compounds are added individually to isolated cultures of ALS-like cells, such as cultures in individual bottles, dishes, plates or wells in a multi-well plate. In other embodiments compounds are added as mixtures of several compounds. In some embodiments, compounds are added as combinatorial libraries or sublibraries of compounds. In some embodiments the identities of the active compounds within a mixture of compounds is determined by deconvolution of data obtained with overlapping sublibraries of compounds. In other embodiments the identity of active compounds is determined by screening of the individual compounds in an active mixture separately or in small groups. The method of detection of the active compounds in a mixture of compounds or in a library is not a critical aspect of the invention.

As demonstrated in Example 1, the use of SOD1-Gly93Ala as the mutant SOD gene causes the formation of aggregates of SOD within transduced rat spinal motor neurons and glial cells, and vacuolization of striatal neurons and glial cells from rat brain. The morphological characteristics can be observed visually by immunocytochemistry, as can any reversal of such morphological characteristics when transduced cells are treated with a potential therapeutic agent or treatment. In other embodiments, the observation of the ALS-like phenotype is automated, for example by computer-assisted image analysis that is able to detect aggregation or vacuolization without human intervention. Such computer-assisted image analysis is particularly preferred in the screening of large numbers of potential therapeutic agents or treatments. In yet other embodiments, SOD1 mutants other than Gly93Ala are used, and the phenotype of cells transduced with these other mutant SOD1 genes may differ from the phenotype associated with SOD1Gly93Ala transduction. Any detectable phenotypic change associated with mutant SOD transduction can be used to assess both whether cells have been transduced to an ALSlike phenotype, and also whether a potential therapeutic agent or treatment is effective at reversing the ALS-like phenotype.

Compounds that can be screened include any compounds that can be provided in the cultures of ALS-like cells. These compounds include, but are not limited to, natural products (either crude mixtures or highly purified components), synthetic compounds, combinatorial libraries, and libraries of known pharmaceutically active compounds. Synthetic compounds can be randomly selected or synthesized specifically for use in treatment of ALS using rational drug design. Combinatorial libraries can be derived from combinatorial synthesis of small molecules or by combinatorial synthesis of polymeric molecules, such as oligonucleotides, oligosaccharides or peptides. Libraries to be screened using the in vitro model system of ALS of the present invention may comprise any number of individual compounds, including 10, 50, 100, 500, 1000, 10,000, 100,000 to 1,000,000 or more. “High throughput screening,” as used herein, refers to screening of more compounds per unit time (e.g. per day) than is possible with the same expenditure of time and effort using assays such as the transgenic mouse SOD1-Gly93Ala model. In various embodiments, high throughput screening refers to screening of 10, 20, 50, 100, 500, 1000, 2000, 5000 or more compounds per day. The model systems of the present invention can also be used to screen treatments that do not involve addition of an agent for the ability of the treatment to reverse or prevent ALS-like phenotype.

Compounds showing efficacy in an in vitro assay of the present invention can then be studied further, e.g. in other in vitro assays, in vivo assays (such as ALS mice, as described supra), or in clinical trials. Such subsequent tests are relatively more labor intensive, time consuming and expensive than the in vitro model system of the instant invention, and thus it would not be practical to screen a large number of compounds using such labor-intensive tests. It is possible, using the in vitro ALS model of the present invention, to reduce the number of candidate compounds sufficiently to make such labor intensive testing practical. In this way the in vitro assay of the present invention makes possible the screening of more compounds than would be practically possible using prior art screening methods, thus increasing the odds of discovering an effective lead compound for treatment or prevention of ALS.

Basic Research Uses of In Vitro Model Systems of ALS

The ALS model systems of the invention not only provide an efficient method for high throughput screening, they also provide a valuable experimental model for studying disease pathogenesis, and defining the basis for the selective vulnerability of motor neurons in ALS. For example, cells in primary culture can be transduced with an AAV virion encoding a mutant SOD1 associated with ALS, and RNA can be harvested from the transduced cells at times from four hours to four days post-transduction. RNA is also obtained from control cells that are either transduced with AAV virions encoding wtSOD1 or cells that are not transduced with any virions. The RNA samples are then subjected to gene expression analysis on an Affymetrix Gene Chip™ to determine which genes are over-expressed, and which are under-expressed, in the ALS model cells compared to the controls. Genes showing differential expression may represent attractive avenues for therapeutic intervention.

In Vivo (Animal) Model Systems of ALS to Screen for Therapeutic Compounds

Recombinant AAV vectors encoding mutant forms of SOD can also be used to create new animal models for ALS. Animals can be administered rAAV vectors (e.g. rAAV virions) encoding a mutant SOD1 gene to produce an ALS-like phenotype. Animals exhibiting an altered phenotype, including phenotypes mimicking the symptoms of ALS, are then used in experiments to test the efficacy of potential therapeutic compounds or treatments. Compounds causing a reversal in the ALS-like phenotype can then be studied further for development as therapeutic agents. Animals that can be used in such ALS models include, but are not limited to, mice, rats and non-human primates.

Examples of specific embodiments of the present invention are provided below. The examples are offered for illustrative purposes only, and are not intended to limit the scope of the present invention in any way.

### EXAMPLE 1

### In Vitro Model of ALS in Primary Rat Motor Neural Cultures

An in vitro model system for ALS is constructed as follows. Two AAV vectors are created by cloning either the human SOD1 wild-type gene (hSOD1wt) or a mutant SOD1 gene (hSOD1-Gly93Ala) gene into an AAV-2-derived vector comprising two AAV inverted terminal repeats (ITRs) such that expression of the SOD gene is directed by the chicken beta actin promoter.

The resulting rAAV2-SOD1-Gly93Ala vector is then packaged into AAV-2 virions (see e.g. U.S. Pat. Nos. 6,001,650, and 6,004,797) and used to transduce primary rat motor neural cultures. Fluorescence microscopy 3-5 days post-transduction reveals that transduced cells exhibit pathological changes characteristic of ALS, such as abnormal distribution of mutant SOD protein in punctate aggregates in most mutant SOD-expressing motor neurons, extensions of perikaryal cytoplasm and swelling of motor neural processes, apoptotic death of motor neurons and activation of astrocytes. rAAV2-SOD1 wt is used as a control in the ALS model of the invention since transduction with that vector does not cause any ALS-associated phenotypic changes in the target cells.

The presence of SOD was visualized via immunohistochemistry in motor neurons in a primary culture from rat spinal cord approximately 3-5 days after transduction with AAV2 vectors encoding pVm-WTSOD or pVm-G93ASOD, respectively. Immunohostochemistry was performed with a mouse anti-SOD1 IgG primary antibody and an Alexa-594-labeled goat anti-mouse IgG secondary antibody. Neurons transduced with pVm-G93ASOD show aggregated SOD1 that is not observed in neurons transduced with pVm-WTSOD.

The presence of SOD was visualized via immunohistochemistry in glial cells in a primary culture from rat spinal cord approximately 3-5 days after transduction with AAV2 vectors encoding pVm-WTSOD or pVm-G93ASOD; respectively. Glial cells transduced with pVm-G93ASOD show aggregated SOD1 that is not observed in glial cells transduced with pVm-WTSOD.

Separate experiments are performed, analogously with those described above, but using AAV vectors derived from AAV-5 and AAV-6, i.e. AAV vectors in which the ITRs are derived from AAV-5 and AAV-6, respectively. rAAV-5 and rAAV-6 virions are then produced using packaging systems that use AAV-5 or AAV-6 capsid protein genes, respectively.

The presence of SOD was visualized via immunohistochemistry in motor neurons in a primary culture from rat spinal cord approximately 3-5 days after transduction with AAV-6 vectors encoding pVm-WTSOD or pVmG93ASOD. Neurons transduced with pVm-G93ASOD show aggregated SOD1 that is not observed in neurons transduced with wtSOD1.

Results show that genes delivered using rAAV-6 virions are predominantly expressed in neurons while genes delivered using rAAV-2 virions are expressed both in neurons and in glia (as discussed supra). Genes delivered using rAAV-5 virions are expressed only in glia, and rAAV-5 virion transduction did not cause pathology in motor neurons.

The phenotypic effects of rAAV2-SOD transduction on motor neurons are measured again 6-7 days and 12 days post-transduction. The results show progression of aggregation over time leading to cell death at 12 days post-transduction. The presence of SOD was visualized via immunohistochemistry in motor neurons in a primary culture from rat spinal cord approximately 6-7 days after transduction with AAV2 vectors encoding pVm-G93ASOD. Aggregation of SOD1 in neurons transduced with pVm-G93ASOD is more extensive than it was at 3-5 days after transduction, illustrating the progression of damage over time. The presence of SOD was visualized via immunohistochemistry in motor neurons in a primary culture from rat, spinal cord approximately 12 days after transduction with AAV2 vectors encoding pVm-G93ASOD. The results show that SOD1-Gly93Ala is toxic to the cells, and that it eventually kills them.

The morphology of motor neurons 6-7 days after transduction with rAAV-SOD1-G93A was evaluated and compared to the morphology of motor neurons from an ALS patient. The presence of SOD was visualized via immunohistochemistry in a primary culture from rat spinal cord approximately 6-7 days after transduction with AAV2 vectors encoding pVm-G93ASOD. rAAV-SOD1-G93A-transduced cells in culture exhibit the same focal axonal swelling characteristic of ALS motor neurons, suggesting that the in vitro ALS model system of the present invention mimics the disease state. Neurons transduced with pVm-G93ASOD show extensions of perikaryal cytoplasm and swelling of motor neural processes at day 6-7 that are similar to those seen in motor neurons from ALS patients.

The same virions used to transduce rat spinal cord cultures (supra) are also used to transduce striatal neurons of primary rat brain cultures. Immunocytochemistry of the transduced cells 3-5 days post-transduction shows that striatal neurons exhibit vacuolization when transduced with vectors expressing SOD1-G93A. A similar phenotype is observed when glial cells from a primary culture of rat brain are transduced with AAV vectors expressing SOD1-G93A. The glial cells from the brain exhibit vacuolization when transduced with pVm-G93ASOD, as contrasted to the aggregation observed with similar treatment of spinal glial cells (discussed supra). This phenotype observed in the brain cells contrasts with the SOD1 aggregate formation observed in transduced spinal motor neurons and glia.

Experiments confirm that the cells expressing SOD1-G93A are in fact motor neurons. Immunocytochemistry was performed on cells in a primary culture of rat spinal cord approximately 5-6 days post-transduction with rAAV-SOD1wt or rAAV-SOD1-G93A. A first experiment involved immunocytochemical detection of both SOD and neurofilament light (NF-L). Both SOD and neurofilament light (NF-L) were detected in motor neurons in a primary culture from rat spinal cord approximately 3-5 days after transduction with AAV2 vectors encoding pVm-WTSOD or pVm-G93ASOD, respectively. NF-L is a specific neural marker. Staining of both SOD and NF-L in the same cell confirms that the cell is a neuron. A second experiment involves immunocytochemical detection of both SOD and choline acetyltransferase (ChAT). Both SOD and choline acetyltransferase (ChAT) were detected in motor neurons in a primary culture from rat spinal cord approximately 3-5 days after transduction with AAV-2 vectors encoding pVm-WTSOD or pVm-G93ASOD, respectively. ChAT is a specific marker for motor neurons. Staining of both SOD and ChAT in the same cell confirms that the cell is a motor neuron. These experiments demonstrate SOD expression within the cells expressing proteins that are characteristic of motor neurons.

Fluorescence microscopy indicates that approximately 90% of all cells in the primary rat motor neural culture are transduced. The high efficiency of transduction using rAAV-SOD1-Gly93Ala provides a large number of cells suitable for use in the assay with relatively little labor, simply by adding the appropriate number of viral particles and incubating. In this example cells are transduced with 100,000 rAAV-SOD1-Gly93Ala particles per cell, i.e. the multiplicity of infection (MOI) is 105. Other experiments (not shown) show that an MOI as low as 1000 is equally effective in providing maximal (90%) transduction. The high number of transduced cells makes it possible to observe the effects of any given compound of interest in a statistically significant number of different cells, and thus enable statistically robust conclusions. This is to be contrasted with prior methods involving micro-injection of mutant SOD encoding plasmids into individual cells, which as a practical matter is not able to provide a statistically meaningful number of cells for high-throughput screening.

This epigenetic model, which employs a viral vector transducing a large number of motor neurons and other cells simultaneously, may facilitate studies of the molecular pathology of ALS, the generation of new animal models of ALS, and screening for ALS drugs.

Materials and methods for Example 1 are as follow.

Construction of pVm-wtSOD and pVm-G93A Sod Plasmids

Human SOD genes of wild type and G93A mutant are amplified by PCR with the forward primer containing an incorporated HindIII site and the reverse primer with an incorporated NotI site.

(SEQ ID NO. 2)

5′-AGCTAAGCTTCCACCATGGCGACGAAGGCCGTGTG-3′ (HC#
146)

(SEQ ID NO. 3)

5′-ATATGCGGCCGCTTATTGGGCGATCCCAATIACACCA-3′. (HC#
147)

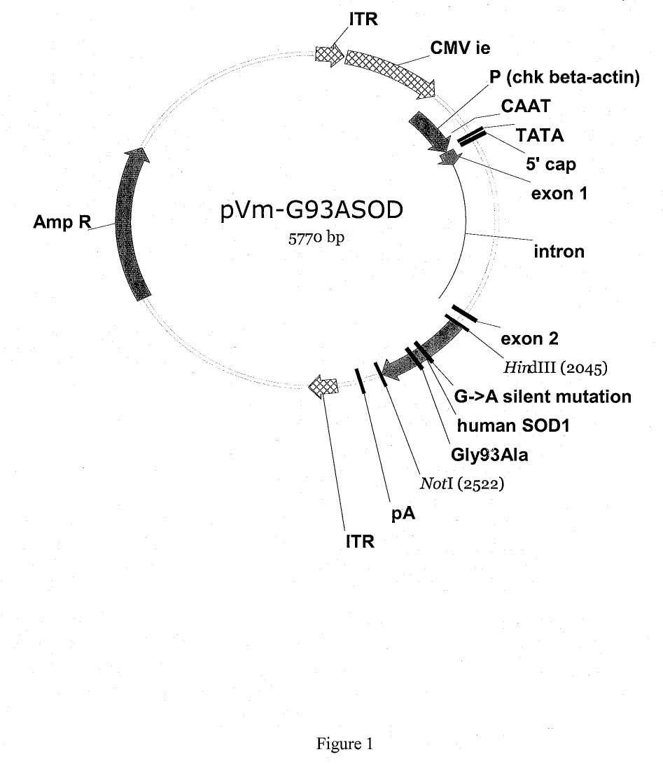

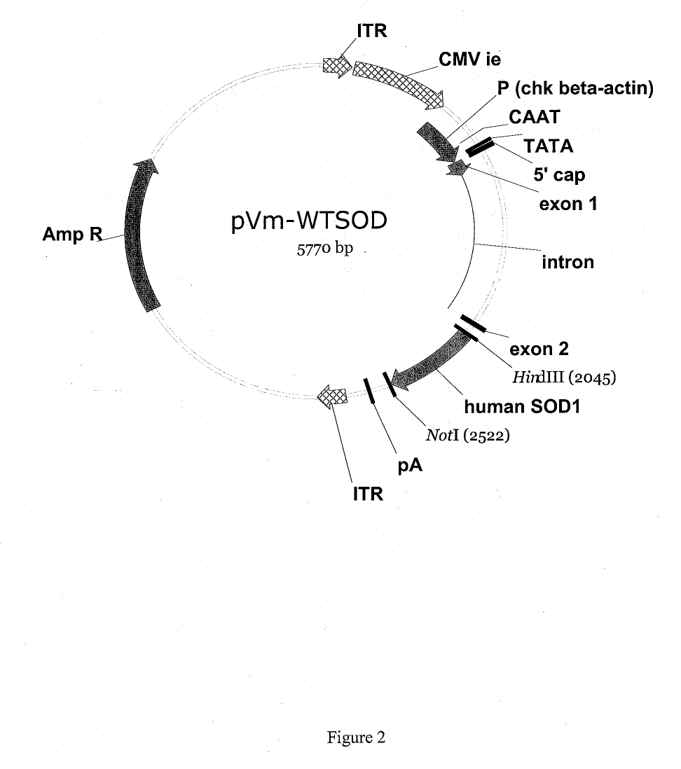

The PCR products are digested with HindIII and NotI restriction enzymes and cloned into the HindIII and NotI sites of plasmid F101, and the genes are placed under the control of chicken beta actin promoter and flanked by both AAV ITRs to create the plasmids F101-wtSOD and F101-G93A. wtSOD and G93A, together with the chicken beta actin promoter, are cut out of F101-wtSOD and F101-G93A with BglII and NotI and cloned into the SphI and NcoI sites of pVm-LacZ using SphI-BglII and NotI-NcoI linkers. The constructs are verified by sequencing analysis and named pVm-wtSOD (FIG. 2) and pVm-G93ASOD (FIG. 1). The plasmids are then amplified and used to transfect 293 cells to produce AAV vectors.

Primary Neural Cultures

Primary cultures of dissociated spinal cord or brain are prepared from embryonic day 15 Sprague-Dawley rat embryos. Dissected striatum or spinal cord tissue is minced into small pieces and incubated with trypsin for 30 min. Following dissociation, the tissue is then triturated through a Pasteur pipette and cells are plated at a density of 350,000 (striatal neurons) or 700,000 cells (spinal cord neurons) per well in 12-well culture dishes (Fisher Scientific, Chicago, Ill.) containing round, glass 18 mm coverslips (Fisher Scientific, Chicago, Ill.) coated with poly-D-lysine (Sigma Chemical Co., St. Louis, Mo.). For striatal cultures the medium is neurobasal medium (Invitrogen, Chicago, Ill.) supplemented with 2% B-27, 0.5 mM L-glutamine and 25 mM L-glutamic acid. Cultures are fed once per week in order to maintain cells. For spinal cord cultures the medium is minimum essential medium eagle (EMEM), (ATCC, Manassas, Va.) enriched with 2.5 g D-glucose and supplemented with 2% horse serum, 5% fetal calf serum, 1% penstrep and growth factors. Cultures are fed twice per week to obtain optimal cell growth and stability. Non-neuronal cells are minimized by treating cultures at day 4-6 with 1.4 μg/ml cytosine-B-D-arabinoside (Calbiochem). Cultures are maintained at 37° C. in 5% CO₂. Cells are used in experiments 2-3 weeks after dissociation, in order to allow for motor neuronal growth and differentiation from other neurons.

### Treatment of Cultures

Striatal cultures and spinal cord cultures are incubated with AAV-hSODwt or AAV-hG93A (10⁵ vector genomes (vg) per cell) to achieve maximum expression of vectors.

### Immunocytochemistry

Striatal and spinal cord cells grown on glass coverslips are fixed with 4% paraformaldehyde and permeabilized by 0.05% NP-40. Blocking solution containing 1×PBS, 3% BSA, and 2% goat serum is used. Immunocytochemistry is performed with the following antibodies: Neurofilament-L (NF-L; AB1983, 1:100, Chemicon Inc.), Choline Acetyltransferase (ChAT; AB 143 and MAB305 1:10, Chemicon Inc), Superoxide Dismutase (SOD; 52 1:300, Sigma Chemical Co., St. Louis, Mo.), Glial Fibrillary Acidic Protein (GFAP: AB5804, 1:500, Chemicon mc). All antibodies are diluted in blocking solution. Antibody distribution is visualized by epifluorescence microscopy after incubation with secondary antibodies: anti-mouse/anti-rabbit/anti-rat IgG conjugated to Alexa Fluor 488 (green) or Alexa Fluor 594 (red), diluted 1:200 (Molecular Probes).

### EXAMPLE 2

### Evaluation of an IL-10 Peptide as a Candidate for Treatment of ALS

The value of the in vitro ALS model system of Example 1 of the invention is illustrated by an assay to evaluate the effect of an IL-10 derived peptide on ALS.

Oligopeptide manufacture is achieved by solid-phase synthesis methods known to those skilled in the Art. Analysis of the synthesized oligopeptides includes electrospray mass spectrometry, high performance liquid chromatography, and visual appearance of the purified product. The oligopeptide(s) are prepared in water for injection at 1 mg/ml. An example of a proper IL-10-derived peptide (U.S. Pat. No. 6,159,937) and a ‘scrambled’ control peptide are provided in Table 2. Peptide sequences are provided in the conventional N→C terminal direction. Amino acids are named using the three-letter nomenclature.

TABLE 2

| | |
|---|---|
| Human IL-10 peptide | |
| Ala-Tyr-Met-Thr-Met-Lys-Ile-Arg-Asn | (SEQ ID NO. 4) |
| Scrambled′ peptide | |
| Arg-Ile-Lys-Asn-Met-Ala-Thr-Tyr-Met | (SEQ ID NO. 5) |

Although an exemplary peptide sequence is provided in Table 2, it would be clear to one of skill in the art that various modifications or substitutions could be made to the listed sequence which would retain, and perhaps improve, the efficacy of the peptide in treatment of neuropathic pain or neurodegenerative disease, or improve its pharmacologic properties. Such sequence variants could be tested in one or more of the models described in the following examples to assess their therapeutic efficacy.

For example, IL-10 sequences from non-human species could be used to obtain IL-10-derived peptide sequences differing from the human-derived IL-10 peptide, which non-human IL-10-derived peptides may exhibit improved properties compared to the human-derived sequence. Alternatively, variants can be designed by inspection using known empirical parameters familiar to those of skill in the art of therapeutic peptides. Additionally, rational drug design can be used to design a sequence variant that would be expected to exhibit increased efficacy, which rational drug design can be based on analysis of the three dimensional structure of an IL-10, an IL-10 receptor, or a complex of IL-10 with a receptor.

Primary cultures of rat spinal cord are incubated with rAAV2-SOD1-G93A virions to produce a culture of cells comprising one or more transduced motor neuron cells expressing SOD1-G93A. The IL-10 and scrambled peptides described above are added (in triplicate) to tissue culture plates containing cultures of rAAV-SOD1-G93A transduced cells at various times after transduction. Control plates of transduced cells are not treated with any peptide. SOD immunocytochemistry is performed as a function of time after peptide addition to determine whether the IL-10 peptide, the scrambled peptide, or both ameliorate the phenotypic effects of SOD1-G93A expression (i.e. intracellular SOD aggregate formation or cell death).

If decreased aggregation or cell death is observed in cultures treated with either or both peptides, the peptide giving positive results in the in vitro assay of the present invention is then subjected to more extensive study (e.g. in vivo studies in ALS mice). Positive results in the in vitro assays of the instant invention include reduction in SOD aggregate formation or cell death by 10, 20, 30, 40, 50, 60, 70, 80, 90, 95, 98% or more. Efficacy in treatment or prevention of ALS is ultimately confirmed through clinical trials in human subjects.

In other experiments, 500 sequence variants of the IL-10 peptide shown above are synthesized and assayed for activity in reducing ALS-like phenotype in rAAV-SOD1-G93A-transduced motor neurons. The peptides showing the greatest activity are studied further for efficacy in treatment or prevention of ALS.

Representative, non-limiting examples of other IL-10 sequences for use with the present invention include the sequences described in NCBI accession numbers NM000572, U63015, AF418271, AF247603, AF247604, AF24760.6, AF247605, AY029171, UL16720 (all human sequences), NM012854, L02926, X60675 (rat); NM010548, AF307012, M37897, M84340 (all mouse sequences), U38200 (equine); U39569, AF060520 (feline sequences); U00799 (bovine); U11421, Z29362 (ovine sequences); L26031, L26029 (macaque sequences); AF294758 (monkey); U33843 (canine); AFO8 8887, AF06805 8 (rabbit sequences); AFO 12909, AF 120030 (woodchuck sequences); AF026277 (possum); AF0975 10 (guinea pig); U111767 (deer); L37781 (gerbil); AB107649 (llama and camel).

### EXAMPLE 3

### Evaluation of GDNF Peptides as a Candidate for Treatment of ALS

The value of the in vitro ALS model system of Example 1 of the invention is further illustrated by an assay to evaluate the effect of a glial cell derived neurotrophic factor (GDNF) peptide on ALS. Experiments are performed essentially as described in Example 2 except that a peptide derived from GDNF, and a scrambled version thereof are provided rather than IL-10 peptide. If a GDNF peptide shows a positive result in the assay (i.e. if there is a reduction in ALS-like phenotypic characteristics of transduced motor neurons after treatment with the peptide), this peptide can be studied further to confirm its efficacy in the treatment or prevention of ALS.

In other experiments, 500 different GDNF-derived peptides are synthesized and assayed for activity in reducing ALS-like phenotype in rAAV-SOD1-G93A-transduced motor neurons. The peptides showing the greatest activity are studied further for efficacy in treatment or prevention of ALS.

GDNF peptides for use in the present invention may be derived from a number of known GDNF sequences, including those disclosed in U.S. Pat. Nos. 6,221,376 and 6,363,319, incorporated herein by reference in their entireties, and Lin et al., Science (1993) 260:1130-1132 for rat and human sequences, as well as NCBI accession numbers AY052832, AJ001896, AF053748, AF063586 and L19063 for human sequences; NCBI accession numbers AF184922, AF497634, X92495, NM019139 for rat sequences; NCBI accession number AF5 16767 for a giant panda sequence; NCBI accession numbers XM122804, NM010275, D88351S1, D49921, U36449, U37459, U66195 for mouse sequences; NCBI accession number AF469665 for a Nipponia nippon sequence; NCBI accession number AF106678 for a Macaca mulatta sequence; and NCBI accession numbers NM13 1732 and AF329853 for zebrafish sequences. GDNF sequences are also disclosed at U.S. Patent Application No. 2003/0161814.

These and other embodiments of the subject invention will readily occur to those of skill in the art in view of the disclosure herein. Although preferred embodiments of the subject invention have been described in some detail, it is understood that variations can be made without departing from the spirit and the scope of the invention as defined herein. All publications, patents and patent applications cited herein are hereby incorporated by reference in their entireties.

We claim:

1. A recombinant adeno-associated virus (AAV) vector comprising: at least one AAV inverted terminal repeat (ITR) sequence; and a gene encoding superoxide dismutase (SOD).

2. The vector of claim 1 wherein the SOD is SOD1.

3. The vector of claim 2 wherein the SOD1 contains a mutation associated with ALS.

4. The vector of claim 3 wherein the SOD1 contains a Gly93Ala mutation.

5. The vector of claim 1 wherein the AAV vector is a plasmid.

6. The vector of claim 1 wherein the AAV vector is an AAV virion.

7. The vector of claim 6, wherein the AAV virion is derived from AAV-2.

8. The vector of claim 6, wherein the AAV virion is derived from AAV-5.

9. The vector of claim 6, wherein the AAV virion is derived from AAV-6.

10. The method of treating a subject comprising: administering to said subject an AAV vector encoding SOD.

11. The method of claim 10 wherein the subject has a disorder selected from the group consisting of ALS, Parkinson's disease, Huntington's disease, Alzheimer's disease, Down syndrome, rheumatoid arthritis, Crohn's disease, Peyronie's disease, ulcerative colitis, niacular degeneration, retinitis pigmentosa, cataracts, cerebral ischemia, myocardial infarct, brain trauma, spinal cord trauma, reperfusion damage, schizophrenia, epilepsy, human leukemia and diabetes.

12. A method of screening compounds for efficacy in the treatment or prevention of amyotrophic lateral sclerosis (ALS) comprising:
transducing a cell with an AAV vector encoding SOD1 containing a mutation associated with ALS to produce a transduced cell, wherein said transduced cell exhibits phenotypic characteristics associated with ALS; exposing said transduced cell to a compound of interest; determining whether or not the transduced cell exposed to the compound of interest exhibits a reduction of the phenotypic characteristics associated with ALS.

13. An in vitro model for screening compounds for efficacy in the treatment of ALS comprising:
a plurality of cells transduced with an AAV vector encoding an SOD containing a mutation associated with ALS.

14. The model of claim 12 or claim 13, wherein the mutation is Gly93Ala.

15. The model of claim 13, wherein the plurality of cells transduced with the AAV vector comprise at least 80% of all cells in a population of cells in culture.


SEQUENCE LISTING (5 sequences)

SEQ ID NO: 1 - protein, 153 aa, Homo sapiens
ATKAVCVLKGDGPVQGIINFEQKESNGPVKVWGSIKGLTEGLHGFHVHEFGDNTAGCTSAGPHFNPLSRKHGGPKDEERHVGDLGNVTADKDGVADVSIEDSVISLSGDHCIIGRTLVVHEKADDLGKGGNEESTKTGNAGSRLACGVIGIAQ

SEQ ID NO: 2 - DNA, 35 bp, Artificial
  forward primer
agctaagcttccaccatggcgacgaaggccgtgtg

SEQ ID NO: 3 - DNA, 37 bp, Artificial
  reverse primer
atatgcggccgcttattgggcgatcccaattacacca

SEQ ID NO: 4 - protein, 9 aa, Homo sapiens
AYMTMKIRN

SEQ ID NO: 5 - protein, 9 aa, Artificial
  scrambled human IL-10 peptide
RIKNMATYM